(19) United States
(12) Patent Application Publication
(10) Pub. No.: US 2025/0020706 A1
(43) Pub. Date: Jan. 16, 2025

(54) METHOD FOR DETERMINING THE DURATION OF DISPERSION HARDENING

(71) Applicant: HERAEUS PRECIOUS METALS GMBH & CO. KG, Hanau (DE)

(72) Inventors: Matthias WEGNER, Hanau (DE); Jochen SCHUSTER, Hanau (DE); Luca KROHN, Hanau (DE)

(21) Appl. No.: 18/769,627

(22) Filed: Jul. 11, 2024

(30) Foreign Application Priority Data
Jul. 12, 2023 (EP) 23185034.8

(51) Int. Cl. G01R 27/02; G01R 19/00

(52) U.S. Cl. CPC G01R 27/02; G01R 19/0084; G01R 19/0092

(57) ABSTRACT

A measurement arrangement for determining the duration of thermal dispersion hardening of a metal sheet wherein the metal sheet is connected to a current or voltage source via a first electrical contact face and a second electrical contact face and to a voltage or current measurement device via a third electrical contact face and a fourth electrical contact face, wherein the first electrical contact face lies completely within a face consisting of the intersecting faces of a front side, a rear side, and a side faces with an imaginary first cylinder, wherein the cylinder axis of the imaginary first cylinder passes through one of the corners of the front side the second electrical contact face lies completely within a face consisting of the intersecting faces of the front side, the rear side, and the side faces with an imaginary second cylinder.

### CROSS-REFERENCE TO RELATED APPLICATIONS

This application claims priority pursuant to 35 U.S.C. 119 (a) to European Application No. 23185034.8, filed Jul. 12, 2023, which application is incorporated herein by reference in its entirety.

### FIELD

The present disclosure relates to measuring an electrical variable of a metal-containing shaped body during thermal dispersion hardening of the metal-containing shaped body.

### BACKGROUND

It is generally known that the mechanical strength of metal materials can be significantly increased by dispersion hardening, in particular for high temperature applications. Dispersion-hardened metals contain non-metal particles, for example oxide particles, which are dispersed in a metal matrix. When dispersion hardening is performed using oxide particles, the metals obtained thereby are also referred to as “ODS” metals (ODS: “oxide dispersion strengthened”).

In this context, reference can be made to the following textbook references by way of example: G. Gottstein, “Materialwissenschaft und Werkstofftechnik—Physikalische Grundlagen,” 4th edition, 2014, SpringerVieweg, page 276 (“Dispersionshärtung”); A. C. Reardon (Editor), “Metallurgy for the Non-Metallurgist,” 2nd edition, 2011, ASM International, pp. 69-70 (“Other Important Strengthening Mechanisms—Dispersion Strengthening”); and, W. D. Callister and D. G. Rethwisch, “Materials Science and Engineering—An Introduction,” 10th edition, 2018, Wiley, page 571 (“Dispersion-Strengthened Composites”)

Because the inorganic particles dispersed in the metal matrix are thermally stable even at high temperatures, i.e., for example, they do not dissolve in the surrounding metal matrix even at high temperatures, dispersion-hardened metals are particularly interesting for high temperature applications.

Dispersion hardening can be performed, for example, by powder metallurgical means through mechanical grinding (e.g., grinding of oxidic particles into a metal matrix) with subsequent compaction through a sintering process.

Alternatively, in a melting metallurgy process, a starting alloy can first be produced that contains alloy elements that can be oxidized at a low concentration. By treating the starting alloy in an oxidative atmosphere, the oxidizable alloy elements are converted to oxide particles, and a dispersion-solidified material is obtained containing a metal matrix and oxide particles dispersed therein. This type of dispersion hardening is also referred to as “internal oxidation.”

Dispersion hardening can be carried out with a variety of different metals.

Shaped bodies made of platinum are often used in high temperature processes in which the material has to have high corrosion resistance. For example, platinum-containing components such as stirrers or glass fiber bushings are used in the glass industry. Because platinum has a low mechanical strength at high temperatures, components made of dispersion-hardened platinum alloys are often used for applications in high temperature processes.

The preparation, processing, and physical properties of such dispersion-hardened platinum compositions are known, for example, from the documents EP 3 971 311 B1, GB 1 280 815 A, GB 1 340 076 A, GB 2 082 205 A, EP 0 683 240 A2, EP 0 870 844 A1, EP 0 947 595 A2, EP 1 188 844 A1, EP 1 295 953 A1, EP 1 964 938 A1, U.S. Pat. Nos. 2,636,819 A, 4,507,156 A, DE 23 55 122 A1, WO 81/01013 A1 and WO 2015/082630 A1.

During the production of a dispersion-solidified shaped body, for example, a semi-finished product (e.g., a metal sheet or a tube) is first produced by melting metallurgy means using a starting alloy (e.g., by casting into a mold), optionally brought into the desired shape by shaping processes, and subsequently subjected to a thermal treatment in an oxidative atmosphere so that oxide particles dispersed in the metal matrix can form.

The preparation of dispersion-solidified alloys is a complex and time-consuming process. The oxide particles in the metal matrix are formed by internal oxidation by diffusion of oxygen into the shaped body.

The alloy elements added to the alloy to form the oxide particles should be oxidized as completely as possible throughout the volume of the alloy. Usually, the larger the volume of the material to be hardened, the longer the period of time that is required in order to achieve complete oxidation. This applies in particular to semi-finished products such as metal sheets or tubes that have a significant extent in at least two spatial directions compared to wires.

The degree of oxidation of an alloy can be determined, for example, by determining the oxygen content using quantitative IR spectroscopy. However, this method first requires that a defined sample amount be taken from the material to be examined and then subsequently analyzed spectroscopically using an NDIR sensor. Alternatively, the degree of oxidation of the sample can be examined by area analysis in the metallographic section, which also requires sample preparation that damages the shaped body. In addition, in both cases (i.e., the measurement of the oxygen content by means of IR spectroscopy or area analysis in the metallographic section), the internal oxidation process must be interrupted and only after measurement has taken place can it be decided whether the material is completely oxidized or whether the oxidation process must be continued.

M. Bruncko et al., “In-situ monitoring of internal oxidation of dilute alloys,” Corrosion Science, 49, 2007, pp. 1228-1244, describe a method in which, during dispersion hardening of an alloy, the electrical resistance of said alloy was determined via a four-wire measurement in order to draw conclusions about the degree of oxidation of the alloy and the required duration of dispersion hardening. The measurement was carried out on a wire (length: 150 mm; diameter: 0.5 mm) and thus a substantially one-dimensional shaped body.

### SUMMARY

An object of the present invention is to provide a measurement method that, during dispersion hardening of a shaped body having a significant extent in at least two spatial directions, in particular of a metal sheet, tube or rod, allows for reliable conclusions about the degree of oxidation of the shaped body without the dispersion hardening having to be interrupted or the shaped body having to be damaged.

According to a first embodiment of the invention, the object is achieved by a method for measuring an electrical variable of a metal-containing shaped body during thermal dispersion hardening of the metal-containing shaped body, wherein
the metal-containing shaped body is a metal sheet having a front side, a rear side opposite the front side, and side faces that connect the front side and the rear side to one another, wherein
the front side and the rear side in each case have longitudinal edges of which the length L_{longitudinaledge} is at least 50 mm, transverse edges of which the length L_{transverseedge} is at least 50 mm, and corners at which the longitudinal edges and transverse edges meet,
the metal sheet has a first electrical contact face and a second electrical contact face, which are connected to a current and/or voltage source, wherein
the first electrical contact face lies completely within a face consisting of the intersecting faces of the front side, the rear side, and the side faces with an imaginary first cylinder, wherein the cylinder axis of the imaginary first cylinder is perpendicular to the front side of the metal sheet and passes through one of the corners of the front side, the cylinder jacket of the imaginary first cylinder intersects the longitudinal edge of the front side at a distance a1 from the cylinder axis and intersects the transverse edge of the front side at a distance b1 from the cylinder axis, wherein

$$a 1 \leq 0.2 \times {L}_{longitudinaledge} and b 1 \leq 0.2 \times {L}_{transverseedge}$$

wherein
L_{longitudinaledge} is the length of the longitudinal edge and L_{transverseedge} is the length of the transverse edge, which meet at the corner through which the cylinder axis of the imaginary first cylinder passes,
the second electrical contact face lies completely within a face consisting of the intersecting faces of the front side, the rear side, and the side faces with an imaginary second cylinder, wherein the cylinder axis of the imaginary second cylinder is perpendicular to the front side of the metal sheet and passes through the corner of the front side that is at the greatest distance from the corner through which the cylinder axis of the imaginary first cylinder passes, the cylinder jacket of the imaginary second cylinder intersects the longitudinal edge of the front side at a distance a2 from the cylinder axis and intersects the transverse edge of the front side at a distance b2 from the cylinder axis, wherein

$$a 2 \leq 0.2 \times {L}_{longitudinaledge} and b 2 \leq 0.2 \times {L}_{transverseedge}$$

wherein
L_{longitudinaledge} is the length of the longitudinal edge and L_{transverseedge} is the length of the transverse edge, which meet at the corner through which the cylinder axis of the imaginary second cylinder passes,
the metal sheet has a third electrical contact face and a fourth electrical contact face that are connected to a voltage measurement device when the first and second electrical contact faces are connected to a current source, or connected to a current measurement device when the first and second electrical contact faces are connected to a voltage source,
the metal sheet, which is connected to the current or voltage source via its first and second electrical contact faces and to the voltage or current measurement device via its third and fourth electrical contact faces, is thermally treated so that oxide particles are formed in the metal sheet, and
during the thermal treatment, the electrical resistance or an electrical variable of the metal sheet proportional to the electrical resistance is determined.

### BRIEF DESCRIPTION

Various embodiments are disclosed, by way of example only, with reference to the accompanying schematic drawings in which corresponding reference symbols indicate corresponding parts, in which:

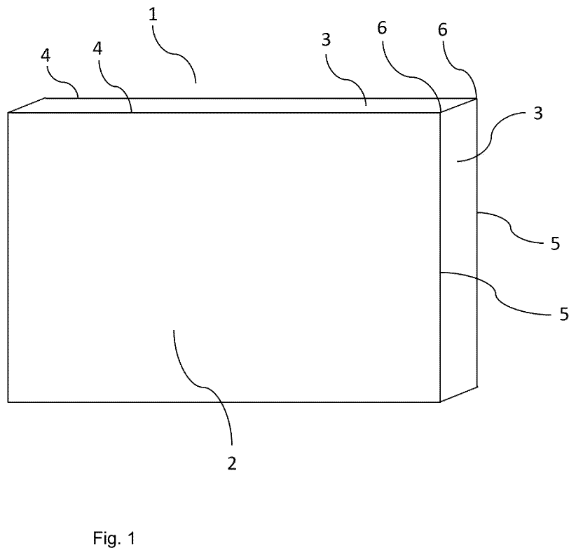

FIG. 1 generally shows a metal sheet;

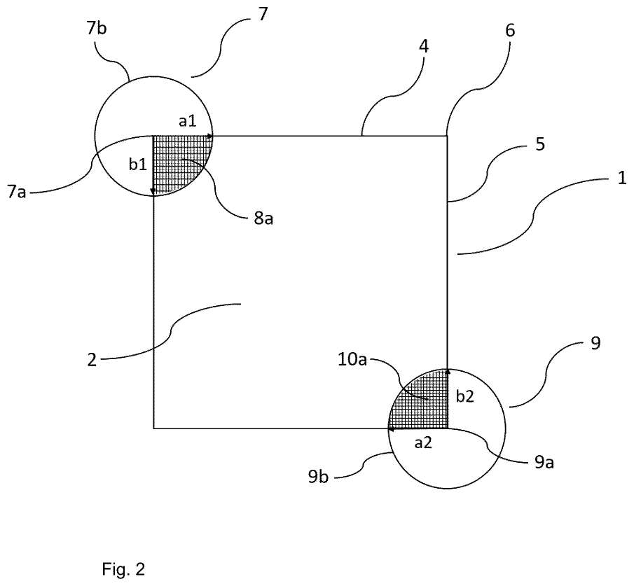

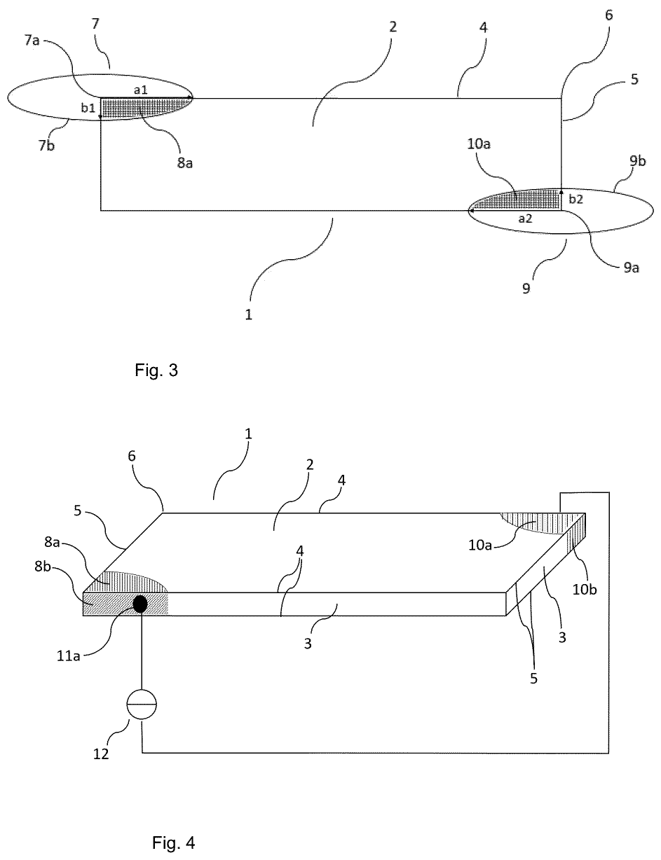

FIGS. 2 and 3 generally show a plan view of the front side of the metal sheet in FIG. 1;

FIG. 4 through 6 generally show perspective views of the metal sheet in FIG. 1, specifically, FIG. 6 is also a schematic representation of the measurement arrangement used in the example according to the invention;

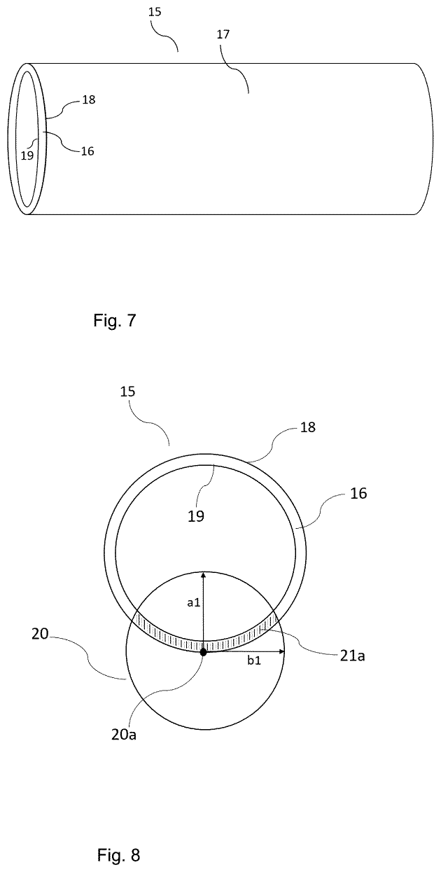

FIG. 7 generally shows exemplary metal tube on which the measuring method according to the invention can be carried out;

FIG. 8 generally shows the metal tube in FIG. 7 in the direction of its longitudinal axis;

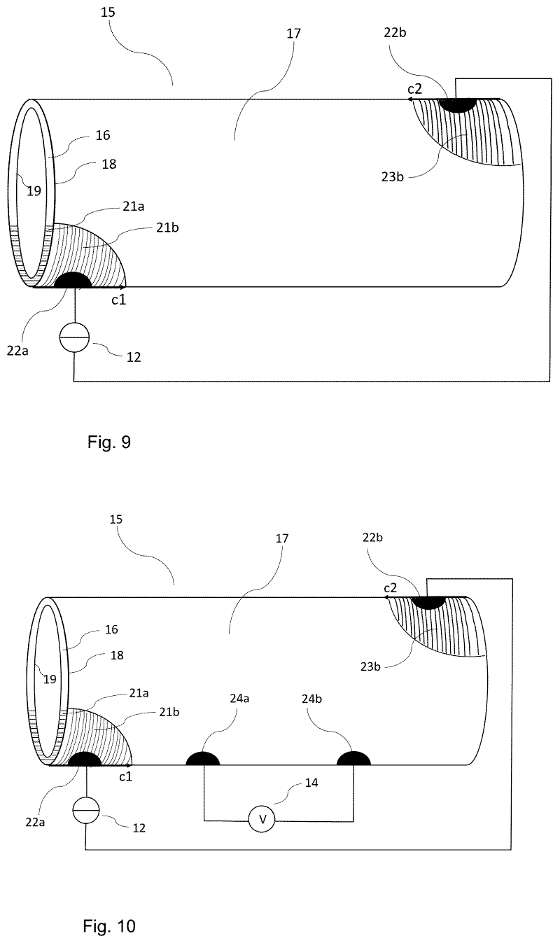

FIGS. 9 and 10 generally show a side-perspective view of the metal tube in FIG. 7; and,

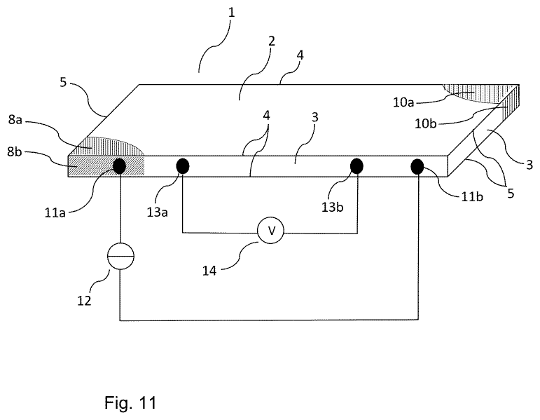

FIG. 11 generally shows a schematic representation of the measurement arrangement of the comparison example.

### DETAILED DESCRIPTION

The method according to the invention is a four-wire measurement. By means of this method, which is known in principle to a person skilled in the art, the electrical (ohmic) resistance or an electrical measured variable of a test body proportional thereto can be determined. However, as described in more detail below, within the scope of the present invention, it is essential where the connections to the current source (or alternatively the voltage source) are attached to the surface of the metal sheet.

Like any metal sheet, the metal sheet on which the measuring method according to the invention is carried out has a front side, a rear side opposite the front side and side faces that connect the front side and the rear side to one another. The front side and the rear side in each case have longitudinal edges, transverse edges, and corners at which the longitudinal edges and transverse edges meet. The longitudinal and transverse edges have lengths of at least 50 mm.

Such an exemplary metal sheet on which the measuring method according to the invention can be carried out is illustrated in FIG. 1.

The metal sheet 1 illustrated in FIG. 1 has a front side 2. The rear side of the metal sheet is located opposite the front side 2. The front side 2 and the rear side of the metal sheet are connected to one another via the side faces 3 and in each case have longitudinal edges 4, transverse edges 5, and corners 6 at which the longitudinal edges 4 and transverse edges 5 meet.

In an exemplary embodiment, the longitudinal edges have a length L_{longitudinaledge} of 50 mm to 2000 mm and/or the transverse edges have a length L_{transverseedge} of 50 mm to 2000 mm. The front side and the rear side of the metal sheet can, for example, have a substantially square shape (i.e. L_{longitudinaledge} and L_{transverseedge} are the same or have at least very similar values) or alternatively have an elongated shape (i.e. L_{longitudinaledge} and L_{transverseedge} have significantly different values). The longitudinal edges and transverse edges of the front side or rear side are, for example, substantially perpendicular to one another (e.g. at an angle of 90°+/−) 10°. The metal sheet has, for example, a thickness of at most 15 mm.

The metal sheet is thus a shaped body that has a significant extent in at least two spatial directions. Accordingly, there are in principle many possibilities for contacting the metal sheet to the current source (or alternatively the voltage source) in the context of a four-wire measurement to determine its electrical resistance.

In the method of the present invention, the four-wire measurement is carried out during the dispersion hardening of the metal sheet. In the context of the present invention, it has been recognized that more reliable conclusions about the degree of oxidation and thus the progress of the dispersion hardening can be drawn from the measurement of the electrical resistance (or an electrical variable proportional to the electrical resistance, such as the electrical voltage or current) of a metal sheet by means of a four-wire measurement if the contacting of the metal sheet with the current source (or alternatively of the voltage source) takes place in such a way that the first electrical contact face is located relatively close to a first corner of the metal sheet, while the second electrical contact face is located as close as possible to the corner that is at the greatest distance relative to the first corner. In the case of a rectangular metal sheet, these are the diagonally opposite corners.

The first electrical contact face lies completely within a face composed of the intersecting faces of the front side, the rear side, and the side faces of the metal sheet with an imaginary first cylinder. The intersecting faces of the front side, rear side, and side faces of the metal sheet with the imaginary first cylinder are the faces of the front side, rear side, and side faces that lie within the imaginary first cylinder. Like any cylinder, this imaginary first cylinder has a cylinder axis and a cylinder jacket. The cylinder axis of the imaginary first cylinder is perpendicular to the front side of the metal sheet and passes through one of the corners at which one of the longitudinal edges and one of the transverse edges of the front side meet. The cylinder jacket of the imaginary first cylinder intersects the longitudinal edge of the front side at a distance a1 from the cylinder axis and intersects the transverse edge of the front side at a distance b1 from the cylinder axis, wherein

$$a 1 \leq 0.2 \times {L}_{longitudinaledge} and b 1 \leq 0.2 \times {L}_{transverseedge}$$

wherein
L_{longitudinaledge} is the length of the longitudinal edge and L_{transverseedge} is the length of the transverse edge, which meet at the corner through which the cylinder axis of the imaginary first cylinder passes.

The imaginary first cylinder can be a circular cylinder or an elliptical cylinder. The distance of a point from the cylinder axis is the length of the shortest connection line between this point and the cylinder axis.

In an exemplary embodiment, a1 and L_{longitudinaledge} and b1 and L_{transverseedge} satisfy the following relationships:

$$a 1 \leq 0.14 \times {L}_{longitudinaledge} and b 1 \leq 0.14 \times {L}_{transverseedge}$$

The second electrical contact face lies completely within a face consisting of the intersecting faces of the front side, the rear side, and the side faces of the metal sheet with an imaginary second cylinder. The intersecting faces of the front side, rear side, and side faces of the metal sheet with the imaginary second cylinder are the faces of the front side, rear side, and side faces that lie within the imaginary second cylinder. The cylinder axis of the imaginary second cylinder is perpendicular to the front side of the metal sheet and passes through the corner of the front side that is at the greatest distance from the corner through which the cylinder axis of the imaginary first cylinder passes. The cylinder jacket of the imaginary second cylinder intersects the longitudinal edge of the front side at a distance a2 from the cylinder axis and intersects the transverse edge of the front side at a distance b2 from the cylinder axis, wherein

$$a 2 \leq 0.2 \times {L}_{longitudinaledge} and b 2 \leq 0.2 \times {L}_{transverseedge}$$

wherein
L_{longitudinaledge} is the length of the longitudinal edge and L_{transverseedge} is the length of the transverse edge, which meet at the corner through which the cylinder axis of the imaginary second cylinder passes.

The imaginary second cylinder can be a circular cylinder or an elliptical cylinder. The distance of a point from the cylinder axis is the length of the shortest connection line between this point and the cylinder axis.

In an exemplary embodiment, a2 and L_{longitudinaledge} and b2 and L_{transverseedge} satisfy the following relationships:

$$a 2 \leq 0.14 \times {L}_{longitudinaledge} and b 2 \leq 0.14 \times {L}_{transverseedge}$$

The imaginary first and second cylinders and the intersecting faces of the front side and side faces of the metal sheet with these imaginary cylinders are illustrated in FIG. 2 (L_{longitudinaledge}=L_{transverseedge}) and FIG. 3 (L_{longitudinaledge}>>L_{transverseedge}).

FIG. 2 is a plan view of the front side 2 of the metal sheet 1, wherein the longitudinal edge 4 and the transverse edge 5 of the front side 2 have the same length and meet at the corners 6. The cylinder axis 7a of the imaginary first cylinder 7 passes through one of the corners 6 and is perpendicular to the front side 2 of the metal sheet 1. The cylinder jacket 7b of the imaginary first cylinder intersects the longitudinal edge 4 at a distance a1 from the cylinder axis 7a and intersects the transverse edge 5 at a distance b1 from the cylinder axis 7a. Because the front side 2 has a square shape (i.e. L_{longitudinaledge}=L_{transverseedge}), the following applies: a1=b1=0.2×L_{longitudinaledge}=0.2×L_{transverseedge}. The front side 2 intersects the imaginary first cylinder 7, resulting in an intersecting face 8a (hatched area in FIG. 2). In addition to this intersecting face 8a, there are other intersecting faces (not shown in FIG. 2) of the side faces and the rear side of the metal sheet with the imaginary first cylinder. The first electrical contact face, via which the metal sheet 1 is connected to the current or voltage source of the measurement arrangement, lies within the face composed of the intersecting face 8a of the front side 2, the intersecting face of the rear side and the intersecting faces of the side faces with the imaginary first cylinder 7 (i.e., consists of these intersecting faces). The cylinder axis 9a of the imaginary second cylinder 9 is perpendicular to the front side 2 of the metal sheet 1 and passes through the corner 6 of the front side 2 that is at the greatest distance from the corner 6 through which the cylinder axis 7a of the imaginary first cylinder 7 passes. The cylinder jacket 9b of the imaginary second cylinder intersects the longitudinal edge 4 at a distance a2 from the cylinder axis 9a and intersects the longitudinal edge 5 at a distance b2 from the cylinder axis 9a. The following also applies to the imaginary second cylinder: a2=b2=0.2×L_{longitudinaledge}=0.2×L_{transverseedge}. The intersecting face 10a of the front side 2 with the imaginary second cylinder is hatched. In addition to this intersecting face, there are other intersecting faces (not shown in FIG. 2) of the side faces and the rear side of the metal sheet with the imaginary second cylinder 9. The second electrical contact face, via which the metal sheet is connected to the current or voltage source of the measurement arrangement, is to be selected within these intersecting faces.

FIG. 3 is a plan view of the front side 2 of the metal sheet 1, wherein the longitudinal edge 4 and the transverse edge 5 of the front side 2 have clearly different lengths and meet at the corners 6. The cylinder jacket 7b of the imaginary first cylinder 7 intersects the longitudinal edge 4 at a distance a1 from the cylinder axis 7a and intersects the longitudinal edge 5 at a distance b1 from the cylinder axis 7a, wherein a1=0.2×L_{longitudinaledge} and b1=0.2×L_{transverseedge}. The front side 2 of the metal sheet 1 intersects the imaginary first cylinder 7 in the intersecting face 8a. The cylinder jacket 9b of the imaginary second cylinder 9 intersects the longitudinal edge 4 at a distance a2 from the cylinder axis 9a and intersects the longitudinal edge 5 at a distance b2 from the cylinder axis 9a, wherein a2=0.2×L_{longitudinaledge} and b2=0.2×L_{transverseedge}. The front side 2 of the metal sheet 1 intersects the imaginary second cylinder in the intersecting face 10a.

As already mentioned above, the electrical contact faces with which the metal sheet is connected to the current or voltage source are to be selected such that they are in each case within a face consisting of the intersecting faces of the front side, the rear side, and the side faces of the metal sheet with the imaginary first or second cylinder.

In the exemplary embodiment illustrated in FIG. 4, the front side 2 of the metal sheet 1 intersects the imaginary first cylinder (not shown in FIG. 4) in the intersecting face 8a and intersects the imaginary second cylinder (not shown in FIG. 4) in the intersecting face 10a. The side faces 3 intersect the first and second imaginary cylinders, among others in the intersecting faces 8b and 10b. The first electrical contact face 11a for connection to the current source 12 is located on the intersecting face 8b, while the second electrical contact face (not shown in FIG. 4) is located on an intersecting face of the opposite side face with the imaginary second cylinder.

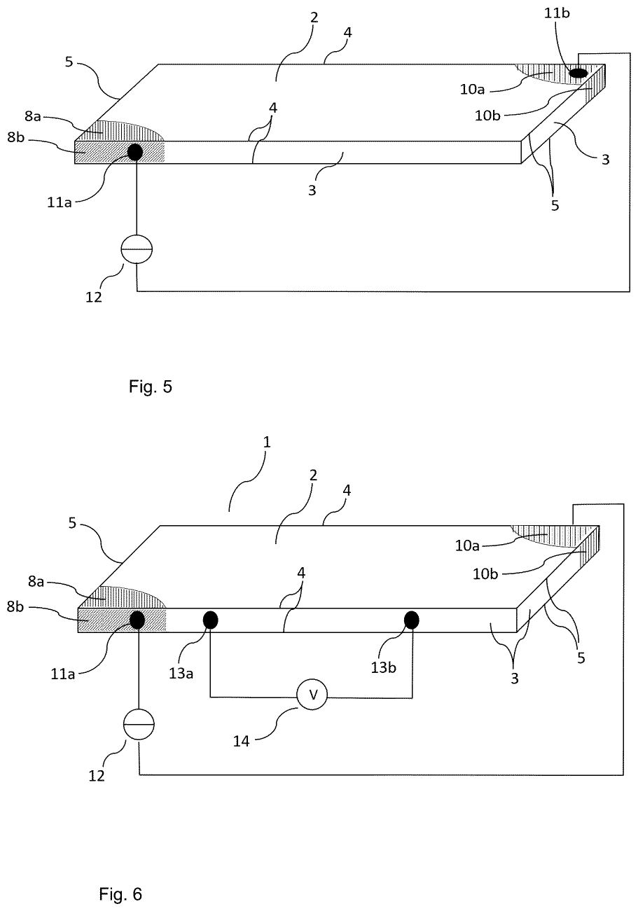

In FIG. 5, the intersecting faces 8a, 8b, 10a, and 10b of the front side 2 and the side faces 3 correspond to the imaginary cylinders of the intersecting faces shown in FIG. 4. Also in accordance with FIG. 4, the first electrical contact face 11a is located within the intersecting face 8b for connection to the current source 12. The second electrical contact face 11b is located within the intersecting face 10a in the exemplary embodiment illustrated in FIG. 5.

A connection is established between the metal sheet and a voltage or current measurement device via a third electrical contact face and a fourth electrical contact face of the metal sheet. When the first and second electrical contact faces of the metal sheet are connected to a current source, the third and fourth electrical contact faces of the metal sheet are connected to a voltage measurement device. When the first and second electrical contact faces of the metal sheet are connected to a voltage source, the third and fourth electrical contact faces of the metal sheet are connected to a current measurement device.

Contact elements known to a person skilled in the art can be used for contacting the electrical contact faces of the metal sheet to the current or voltage source and the voltage or current measurement device. For example, the electrical contact faces are connected to the current or voltage source and to the voltage or current measurement device via terminals (e.g., Kelvin terminals or coil terminals) or contact pins.

Thermal dispersion hardening usually takes place at fairly high temperatures and sometimes over fairly long periods of time. In order to achieve reliable electrical contact between the metal sheet and the current or voltage source under these conditions in a simple and efficient manner, it can be preferred to contact the first or second electrical contact face with an electrical line of the current or voltage source, and to contact the third and fourth electrical contact faces with the electrical lines of the voltage or current measurement device under the effect of gravity of the metal sheet, for example by placing the metal sheet on the electrical lines (for example via its front side or one of its side faces). The metal sheet is, for example, placed on the electrical lines with one of its side faces. In this preferred embodiment, the number of contact terminals required can be significantly reduced.

A person skilled in the art will be able to determine a suitable size of the electrical contact faces on the metal sheet on the basis of his specialist knowledge or possibly by routine testing. For example, the electrical contact faces of the metal sheet each have an area of at least 1 mm².

In the method according to the invention, it is essential that the first electrical contact face and the second electrical contact face of the metal sheet (i.e., the contact faces of the metal sheet via which the metal sheet is connected to the current or voltage source) are selected such that they are in each case within a face consisting of the intersecting faces of the front side, the rear side, and the side faces with the imaginary cylinders. This ensures that the first electrical contact face is located relatively close to one of the corners of the metal sheet, while the second electrical contact face is located as close as possible to the opposite corner.

The positions of the third and fourth electrical contact faces on the metal sheet (i.e., the contact faces of the metal sheet via which the metal sheet is connected to the voltage or current measurement device) can be freely selected. A person skilled in the art can determine suitable positions on the basis of his general specialist knowledge and possibly by routine testing.

For example, the third and fourth electrical contact faces are at a greater distance from the corner through which the cylinder axis of the imaginary first cylinder passes than the first electrical contact face, and

are at a greater distance from the corner through which the cylinder axis of the imaginary second cylinder passes than the second electrical contact face. The distance is the shortest straight connection line between the contact face and the corner.

For example, the third and fourth electrical contact faces are located outside of the intersecting faces of the front side, the rear side, and the side faces with the imaginary first cylinder and outside of the intersecting faces of the front side, the rear side, and the side faces with the imaginary second cylinder.

For example, the third and fourth electrical contact faces are at a distance from one another that is smaller than the distance between the first and second electrical contact faces. The distance is the shortest straight connection line between the two corresponding contact faces.

For example, the first, third and fourth electrical contact faces are on the same side face of the metal sheet, and the second electrical contact face is on another side face, preferably the opposite side face, of the metal sheet. This exemplary embodiment is illustrated in FIG. 6. With regard to the intersecting faces 8a, 8b, 10a, and 10b of the front side 2 and the side faces 3 with the imaginary cylinders and the positions of the first electrical contact face 11a and the second electrical contact face (for the connection to the current source 12), FIG. 6 corresponds to the embodiment illustrated in FIG. 4. The metal sheet 1 is connected to the voltage measurement device 14 via the electrical contact faces 13a and 13b.

The metal sheet, which is connected to the current or voltage source via the first and second electrical contact faces and to the voltage or current measurement device via the third and fourth electrical contact faces, is thermally treated so that oxide particles are formed in the metal sheet.

Suitable metals in the form of alloys that form oxide particles in a thermal treatment in an oxidizing atmosphere (so that a metal matrix and oxide particles dispersed therein are obtained) and that are suitable for dispersion hardening are known to a person skilled in the art.

For example, the metal from which the metal sheet is made is a noble metal alloy that contains one or more non-noble metals. For example, the noble metal alloy contains non-noble metals at a concentration of not more than 1 wt. %, and the balance is one or more noble metals and unavoidable impurities. The non-noble metal is, for example, selected from zirconium, cerium, scandium and yttrium, and the noble metal is, for example, selected from ruthenium, rhodium, gold, palladium, platinum, iridium and osmium. The noble metal alloy contains, for example, a first noble metal as the main element and other noble metals, if any, in a total concentration of not more than 29.95 wt. %, under the proviso that the first noble metal differs from the other noble metals. “Main element” means that this element is present at a higher concentration than any of the other elements.

Suitable temperatures for carrying out the dispersion hardening are known to a person skilled in the art or can optionally be determined by routine testing. For example, the thermal treatment of the metal sheet takes place at a temperature of at least 750° C., for example in the range from 750° C. to 1400° C. or 800° C. to 1200° C.

Dispersion hardening takes place in an oxidizing atmosphere. Suitable oxidizing atmospheres for dispersion hardening of alloys are known to a person skilled in the art. For example, the oxidizing atmosphere is an oxygen-containing atmosphere. For example, the oxygen content of the oxidizing atmosphere is at least 5% by volume, more preferably at least 10% by volume. In an exemplary embodiment, air is used as an oxidizing atmosphere.

During the thermal treatment, the electrical resistance or an electrical variable of the metal sheet proportional to the electrical resistance is determined.

The electrical resistance (also referred to as ohmic resistance) is determined with the known relationship R=U/l, where
I is the current strength of the current supplied by the current source (connected to the first and second electrical contact faces of the metal sheet), and U is the voltage measured with the voltage measurement device (connected to the third and fourth electrical contact faces of the metal sheet), or
U is the voltage applied by the voltage source (connected to the first and second electrical contact faces of the metal sheet), and I is the current strength measured with the current measurement device (connected to the third and fourth electrical contact faces of the metal sheet).

The measured electrical resistance is influenced by the degree of oxidation of the metal sheet. The thermal treatment is ended, for example, when the electrical resistance of the metal sheet has a constant value (e.g. a variation of not more than +/−1%) over a defined duration (e.g. at least 1 hour or at least 4 hours).

Instead of the electrical resistance of the metal sheet, a variable proportional to the electrical resistance, for example the electrical voltage or current strength, can be used to determine the required duration for dispersion hardening. For example, it may be sufficient to measure the voltage or the current strength at the third and fourth contact faces and to end the thermal treatment when the voltage or current strength measured at these contact faces has a constant value (e.g. a variation of not more than +/−1%) over a defined duration (e.g. at least 1 hour or at least 4 hours).

The electrical resistance or the electrical variable proportional thereto can be determined continuously or at defined time intervals, for example.

According to a further embodiment of the present invention, the object is achieved by a method for measuring an electrical variable of a metal-containing shaped body during a thermal dispersion hardening of the metal-containing shaped body, wherein
the metal-containing shaped body is a metal tube or a metal rod, wherein the metal tube or the metal rod
   i. has a length L_{shapedbody} of at least 50 mm and a diameter D_{shapedbody} in the range from 10 mm to 300 mm,
   ii. ends at one of its ends with a first end face and at its opposite end with a second end face, the first and second end faces are connected to one another by a lateral face, the first end face and the second end face are each delimited by an outer boundary line,
the metal tube or the metal rod has a first electrical contact face and a second electrical contact face, and these electrical contact faces are connected to a current or voltage source, wherein
   i. the first electrical contact face lies completely within a face consisting of the intersecting faces of the lateral face and the first end face with an imaginary first ellipsoid, wherein the imaginary first ellipsoid has a center point and semi-axes a1, b1 and c1, wherein the center point lies on the outer boundary line of the first end face of the metal tube or metal rod, the semi-axis a1 is parallel to the longitudinal axis of the metal tube or metal rod and has a length Lₐ₁≤0.2×L_{shapedbody}, and the semi-axes b1 and c1 have a length L_{b1}=L_{c1}≤0.4×D_{shapedbody},
   ii. the second electrical contact face lies completely within a face consisting of the intersecting faces of the lateral face and the second end face with an imaginary second ellipsoid, wherein the imaginary second ellipsoid has a center point and semi-axes a2, b2 and c2, the center point of the imaginary second ellipsoid is the point farthest from the center point of the imaginary first ellipsoid on the outer boundary line of the second end face of the metal tube or metal rod, the semi-axis a2 is parallel to the longitudinal axis of the metal tube or metal rod and has a length Lₐ₂≤0.2×L_{shapedbody}, and the semi-axes b2 and c2 have a length L_{b2}=L_{c2}≤0.4×D_{shapedbody},
the metal tube or the metal rod has a third electrical contact face and a fourth electrical contact face, and these electrical contact faces are connected to a voltage measurement device when the first and second electrical contact faces are connected to a current source, or connected to a current measurement device when the first and second electrical contact faces are connected to a voltage source,
the metal tube or the metal rod, which is connected to the current or voltage source via its first and second electrical contact faces and to the voltage or current measurement device via its third and fourth electrical contact faces, is thermally treated so that oxide particles are formed in the metal tube or metal rod, and
during the thermal treatment of the metal tube or metal rod, its electrical resistance or an electrical variable proportional to the electrical resistance is determined.

Within the scope of the present invention, the term “ellipsoid” also includes a sphere as a limiting case.

The semi-axes a1, b1 and c1 of the imaginary first ellipsoid are usually perpendicular to one another. The semi-axes a2, b2 and c2 of the imaginary second ellipsoid are also usually perpendicular to one another.

If the shaped body is a metal tube, the aforementioned diameter D_{shapedbody} is the outer diameter of the metal tube. In the case of a metal tube, both the first end face and the second end face also have an inner boundary line next to the outer boundary line.

An exemplary metal tube on which the measuring method according to the invention can be carried out is illustrated in FIG. 7.

The metal tube 15 illustrated in FIG. 7 terminates at one of its ends with a first end face 16. The first end face 16 is delimited by an outer boundary line 18 and an inner boundary line 19. The outer boundary line 18 corresponds to the outer circumference of the tube 15, while the inner boundary line 19 corresponds to the inner circumference of the tube 15. At the opposite end, the tube 15 terminates with a second end face (not shown in FIG. 7), which is also delimited by an outer and inner boundary line. The first end face 16 and the opposite second end face are connected to one another by the lateral face 17.

The metal tube or the metal rod has, for example, a length L_{shapedbody} from 50 mm to 3,000 mm.

The metal tube or the metal rod has, for example, a ratio of L_{shapedbody} to D_{shapedbody} of at least 2, more preferably at least 5.

The tube is, for example, a round tube or a polygonal tube (e.g. a rectangular tube).

Selecting a suitable wall thickness for the tube depends, for example, on the planned use of the tube. For example, the tube has a ratio of wall thickness to outer diameter of at least 0.01. The tube has, for example, a wall thickness of not more than 15 mm.

The imaginary ellipsoids and the intersecting faces of the end face and the lateral face of the metal tube with these imaginary ellipsoids are illustrated in FIGS. 8 and 9.

FIG. 8 shows the metal tube 15 in the direction of its longitudinal axis. The center point 20a of the imaginary first ellipsoid 20 is located on the outer boundary line 18 of the first end face 16. The figure shows the semi-axes a1 and b1 of the ellipsoid 20. The first end face 16 intersects the imaginary first ellipsoid 20 in the intersecting face 21a. In addition to this intersecting face 21a, there is also an intersecting face of the lateral face of the cylinder 15 with the imaginary first ellipsoid 20 (not shown in FIG. 8).

In FIG. 9, the first end face 16 and the lateral face 17 of the metal tube 15 intersect the imaginary first ellipsoid (not shown) in the intersecting faces 21a and 21b. The lateral face 17 intersects the imaginary second ellipsoid (not shown) in the intersecting face 23b. The intersecting face of the second end face with the imaginary second ellipsoid is not shown in FIG. 9. The first electrical contact face 22a and the second electrical contact face 22b lie within the intersecting faces 21b and 23b and connect the metal tube 15 to the current source 12.

In an exemplary embodiment, the following relationships apply to the lengths of the semi-axes a1, b1 and c1 of the imaginary first ellipsoid and the lengths of the semi-axes a2, b2, and c2 of the imaginary second ellipsoid:

$${L}_{a 1} \leq 0.14 \times {L}_{shapedbody} ; {L}_{b 1} = {L}_{c 1} \leq 0.25 \times {D}_{shapedbody} ;$$

$${L}_{a 2} \leq 0.14 \times {L}_{shapedbody} ; {L}_{b 2} = {L}_{c 2} \leq 0.25 \times {D}_{shapedbody} ;$$

As in the first embodiment according to the invention (i.e., a metal sheet as a shaped body), in the second embodiment of the method according to the invention, a connection to a voltage or current measurement device is also established via a third electrical contact face and a fourth electrical contact face of the metal shaped body. When the first and second electrical contact faces of the metal rod or metal tube are connected to a current source, the third and fourth electrical contact faces of the metal rod or metal tube are connected to a voltage measurement device. When the first and second electrical contact faces of the metal rod or metal tube are connected to a voltage source, the third and fourth electrical contact faces of the metal rod or metal tube are connected to a current measurement device.

Contact elements known to a person skilled in the art can be used for contacting the electrical contact faces of the metal rod or metal tube with the current or voltage source and the voltage or current measurement device. For example, the electrical contact faces are connected to the current or voltage source and to the voltage or current measurement device via terminals (e.g., Kelvin terminals or coil terminals).

In a preferred embodiment, the first electrical contact face is contacted with an electrical line of the current or voltage source, and the third and fourth electrical contact faces are contacted with the electrical lines of the voltage or current measurement device under the effect of gravity of the metal rod or metal tube, for example by placing the metal rod or the metal tube (for example with its lateral face or one of its end faces) on the electrical lines. In this preferred embodiment, the number of contact terminals required can be significantly reduced.

A person skilled in the art will be able to determine a suitable size of the electrical contact faces on the metal rod or metal tube on the basis of his specialist knowledge and by routine testing. For example, the electrical contact faces of the metal rod or metal tube each have an area of at least 1 mm².

In the method according to the invention, it is essential that the first electrical contact face and the second electrical contact face of the metal rod or metal tube (i.e., the contact faces via which the metal rod or metal tube is connected to the current or voltage source) are selected such that they are in each case within a face consisting of the intersecting faces of the end face and the lateral face with an imaginary first or second ellipsoid. This ensures that the first electrical contact face and the second electrical contact face are at as large a distance from one another as possible.

The intersecting faces of the lateral face and first end face with the imaginary first ellipsoid are the faces of the lateral face and first end face that lie within the imaginary first ellipsoid. The intersecting faces of the lateral face and the second end face with the imaginary second ellipsoid are the faces of the lateral face and the second end face that lie within the imaginary second ellipsoid.

The positions of the third and fourth electrical contact faces on the metal tube or metal rod (i.e., the contact faces of the metal tube or metal rod via which the connection to the voltage or current measurement device is made) can be freely selected. A person skilled in the art can determine suitable positions on the basis of his general specialist knowledge and possibly by routine testing.

For example, the third and fourth electrical contact faces are at a greater distance from the center point of the imaginary first ellipsoid than the first electrical contact face, and

are at a greater distance from the center point of the imaginary second ellipsoid than the second electrical contact face. The distance is the shortest straight connection line between the contact face and the center point of the ellipsoid.

For example, the third and fourth electrical contact faces are located outside the intersecting faces of the end faces and the lateral face with the imaginary ellipsoids.

For example, the third and fourth electrical contact faces are at a distance from one another that is smaller than the distance between the first and second electrical contact faces. The distance is the shortest straight connection line between the two corresponding electrical contact faces.

In FIG. 10, the intersecting faces 21a, 21b, and 23b and the electrical contact faces 22a and 22b (via which the metal tube 15 is connected to the current source 12) correspond to the embodiment illustrated in FIG. 9. The third electrical contact face 24a and the fourth electrical contact face 24b, via which the metal tube 15 is connected to the voltage measurement device 14, are arranged substantially linearly to the first electrical contact face 22a. This can be realized, for example, by placing the metal tube 15 with its lateral face 17 on the electrical lines that are connected to the current source and the voltage measurement device.

As in the first embodiment according to the invention, in the second embodiment of the method according to the invention, the metal shaped body, which is connected to the current or voltage source via its first and second electrical contact faces and to the voltage or current measurement device via its third and fourth electrical contact faces, is also thermally treated, so that oxide particles are formed in the metal tube, and the determination of its electrical resistance or an electrical variable proportional to the electrical resistance takes place during the thermal treatment of the metal shaped body. For further details on these method steps and on suitable metal alloys, reference can thus be made to the above description of the first embodiment according to the invention.

In addition, the present invention relates to a measurement arrangement, containing
a current or voltage source,
a voltage or current measurement device,
a metal-containing shaped body, wherein the metal-containing shaped body is a metal sheet
that has a front side, a rear side opposite the front side, and side faces that connect the front and rear side to one another, wherein
   i. the front side and the rear side in each case have longitudinal edges of which the length L_{longitudinaledge} is at least 50 mm, transverse edges of which the length L_{transverseedge} is at least 50 mm, and corners at which the longitudinal edges and transverse edges meet,
the metal sheet has a first electrical contact face and a second electrical contact face, which are connected to a current or voltage source, wherein
   i. the first electrical contact face lies completely within a face consisting of the intersecting faces of the front side, the rear side, and the side faces with an imaginary first cylinder, wherein the cylinder axis of the imaginary first cylinder is perpendicular to the front side of the metal sheet and passes through one of the corners of the front side, the cylinder jacket of the imaginary first cylinder intersects the longitudinal edge of the front side at a distance a1 from the cylinder axis and intersects the transverse edge of the front side at a distance b1 from the cylinder axis, wherein

$$a 1 \leq 0.2 \times {L}_{longitudinaledge} and b 1 \leq 0.2 \times {L}_{transverseedge}$$

iii, wherein
iv. L_{longitudinaledge} is the length of the longitudinal edge and L_{transverseedge} is the length of the transverse edge, which meet at the corner through which the cylinder axis of the imaginary first cylinder passes,
v. the second electrical contact face lies completely within a face consisting of the intersecting faces of the front side, the rear side, and the side faces with an imaginary second cylinder, wherein the cylinder axis of the imaginary second cylinder is perpendicular to the front side and passes through the corner of the front side that is at the greatest distance from the corner through which the cylinder axis of the imaginary first cylinder passes, the cylinder jacket of the imaginary second cylinder intersects the longitudinal edge of the front side at a distance a2 from the cylinder axis and intersects the transverse edge of the front side at a distance b2 from the cylinder axis, wherein

$$a 2 \leq 0.2 \times {L}_{longitudinaledge} and b 2 \leq 0.2 \times {L}_{transverseedge}$$

vii, wherein
viii. L_{longitudinaledge} is the length of the longitudinal edge and L_{transverseedge} is the length of the transverse edge, which meet at the corner through which the cylinder axis of the imaginary second cylinder passes,
the metal sheet has a third electrical contact face and a fourth electrical contact face, and these electrical contact faces are connected to the voltage measurement device when the first and second electrical contact faces are connected to the current source, or connected to the current measurement device when the first and second electrical contact faces are connected to the voltage source.

The above-described method according to the first embodiment according to the invention can be carried out with this measurement arrangement. For further properties of the metal sheet and the electrical contact faces of the metal sheet, reference can therefore be made to the above description of the first embodiment according to the invention.

In addition, the present invention relates to a measurement arrangement, containing
a current or voltage source,
a voltage or current measurement device,
a metal-containing shaped body, wherein the metal-containing shaped body is a metal tube or a metal rod, wherein the metal tube or the metal rod
   i. has a length L_{shapedbody} of at least 50 mm and a diameter D_{shapedbody} in the range from 10 mm to 300 mm,
   ii. ends at one of its ends with a first end face and at its opposite end with a second end face, the end faces are connected to one another by a lateral face and are each delimited by an outer boundary line,
the metal tube or the metal rod has a first electrical contact face and a second electrical contact face, which are connected to a current or voltage source, wherein
   i. the first electrical contact face lies completely within a face consisting of the intersecting faces of the lateral face and the first end face with an imaginary first ellipsoid, wherein the imaginary first ellipsoid has a center point and semi-axes a1, b1 and c1, wherein the center point lies on the outer boundary line of the first end face of the metal tube or metal rod, the semi-axis a1 is parallel to the longitudinal axis of the metal tube or metal rod and has a length Lₐ₁≤0.2×L_{shapedbody}, and the semi-axes b1 and c1 have a length L_{b1}=L_{c1}≤0.4×D_{shapedbody},
   ii. the second electrical contact face lies completely within a face consisting of the intersecting faces of the lateral face and the second end face with an imaginary second ellipsoid, wherein the imaginary second ellipsoid has a center point and semi-axes a2, b2 and c2, the center point of the imaginary second ellipsoid is the point farthest from the center point of the imaginary first ellipsoid on the outer boundary line of the second end face of the metal tube or metal rod, the semi-axis a2 is parallel to the longitudinal axis of the metal tube or metal rod and has a length Lₐ₂≤0.2×L_{shapedbody}, and the semi-axes b2 and c2 have a length L_{b2}=L_{c2}≤0.4×D_{shapedbody},
the metal tube or the metal rod has a third electrical contact face and a fourth electrical contact face, and these electrical contact faces are connected to a voltage measurement device when the first and second electrical contact faces are connected to a current source, or connected to a current measurement device when the first and second electrical contact faces are connected to a voltage source.

The above-described method according to the second embodiment according to the invention can be carried out with this measurement arrangement. For further properties of the metal tube or metal rod and the electrical contact faces of the metal tube or metal rod, reference can therefore be made to the above description of the second embodiment according to the invention.

The present invention also relates to the use of the above-described measurement arrangements for determining the duration of a thermal dispersion hardening of the metal sheet, metal rod or metal tube.

The present invention is explained in more detail with reference to the following examples.

### Examples

Two starting metal sheets were produced that had matching dimensions and a matching chemical composition.
L_{longitudinaledge} (i.e., length of the longitudinal edge of the metal sheets): 150 mm
L_{transverseedge} (i.e., length of the transverse edge of the metal sheets): 120 mm

A platinum alloy, as described in Example 3 of EP 3971311 B1, was used to produce the two starting sheets.

Each of the starting sheets with matching chemical compositions was then subjected to thermal dispersion hardening under identical conditions in an oxidative atmosphere. Dispersion hardening took place in each case in an air atmosphere at a temperature of 1,000° C.

The aim of dispersion hardening is to oxidize the non-noble metals in the alloy as completely as possible (with formation of oxide particles dispersed in the noble metal matrix).

For both metal sheets, the electrical resistance was determined as a function of time during dispersion hardening using a four-wire measurement. However, as described in more detail below, different measurement arrangements have been used for the four-wire measurement.

The measurement arrangements of the comparison example and of the example according to the invention differed in the relative arrangement of the first and second electrical contact faces relative to one another.

In the measurement arrangement of the comparison example, the first electrical contact face was on one of the side faces close to a first corner of the metal sheet, and the second electrical contact face was on the same side face close to the corner adjacent to the first corner.
Distance from the first electrical contact face to the first corner: 15 mm
Distance from the second electrical contact face to the corner adjacent to the first corner: 15 mm

In the measurement arrangement of the example according to the invention, the position of the second electrical contact face was changed such that it was located on the opposite side face in the vicinity of the corner opposite to the first corner.
Distance from the second electrical contact face to the opposite corner: 15 mm

FIG. 6 is a schematic representation of the measurement arrangement used in the example according to the invention.

FIG. 11 is a schematic representation of the measurement arrangement of the comparison example.

In accordance with FIG. 6, FIG. 11 shows a metal sheet 1 with a front side 2, side faces 3, longitudinal edges 4, transverse edges 5 and intersecting faces 8a, 8b, 10a, 10b of the front side 2 and side faces 3 with the imaginary cylinders. The voltage source 14 is connected to the metal sheet 1 via the third and fourth electrical contact faces 13a and 13b. The current source 12 is connected to the metal sheet via the first and second electrical contact faces 11a and 11b. In contrast to the example according to the invention, in the comparison example, the second electrical contact face 11b is not located on any of the intersecting faces of the front side, the rear side or the side faces of the metal sheet with the imaginary second cylinder.

In both cases, the thermal treatment was ended if the electrical resistance had a constant value for a period of 2 hours.

The measurement arrangement according to the invention resulted in a constant resistance value after 144 hours.

The measurement arrangement of the comparison example resulted in a constant resistance value after 60 hours.

For both metal sheets, the oxygen content was determined at the end of the dispersion hardening by quantitative IR spectroscopy using a device from LECO (ONH836). Further details regarding this method can be found in EP 3 971 311 B1. If the chemical composition of the alloy is known, the degree of oxidation can be calculated from the measured oxygen. The degree of oxidation indicates the proportion at which the non-noble metals have been converted to the corresponding oxides during dispersion hardening. A value that is as close as possible to 100% would be desirable.

The results are summarized in the following table:

| | Time [h] until a resistance | |
|---|---|---|
| | value that is constant | Degree of |
| | for at least 2 hours | oxidation [%] |
| Measurement | 60 | 50 |
| arrangement of the | | |
| comparison example | | |
| Measurement | 144 | 100 |
| arrangement of the | | |
| example according | | |
| to the invention | | |

The data shows that the measurement arrangement according to the invention allows the time required for dispersion hardening to be determined significantly more reliably.

### LIST OF REFERENCE SIGNS

1 Metal sheet
2 Front side of the metal sheet
3 Side faces of the metal sheet
4 Longitudinal edges of the front and rear sides of the metal sheet
5 Transverse edges of the front and rear sides of the metal sheet
6 Corners of the metal sheet
7 Imaginary first cylinder
7a Cylinder axis of the imaginary first cylinder
7b Cylinder jacket of the imaginary first cylinder
8a, 8b Intersecting faces of the front side and side faces of the metal sheet with the imaginary first cylinder
9 Imaginary second cylinder
9a Cylinder axis of the imaginary second cylinder
9b Cylinder jacket of the imaginary second cylinder
10a, 10b Intersecting faces of the front side and side faces of the metal sheet with the imaginary second cylinder
11a First electrical contact face of the metal sheet
11b Second electrical contact face of the metal sheet
12 Current or voltage source
13a, 13b Third and fourth electrical contact faces of the metal sheet
14 Voltage or current measurement device
15 Metal tube
16 First end face of the metal tube
17 Lateral face of the metal tube
18 Outer boundary line of the first end face
19 Inner boundary line of the first end face
20 Imaginary first ellipsoid
20a Center point of the imaginary first ellipsoid
21a, 21b Intersecting faces of the first end face and of the lateral face with the imaginary first ellipsoid
22a First electrical contact face of the metal tube
22b Second electrical contact face of the metal tube
23b Intersecting face of the lateral face with the imaginary second ellipsoid
24a. 24b Third and fourth electrical contact faces of the metal tube

1. A method for measuring an electrical variable of a metal-containing shaped body during thermal dispersion hardening of the metal-containing shaped body, wherein
the metal-containing shaped body is a metal sheet having a front side, a rear side opposite the front side, and side faces that connect the front side and the rear side to one another, wherein
the front side and the rear side in each case have longitudinal edges of which the length L_{longitudinaledge} is at least 50 mm, transverse edges of which the length L_{transverseedge} is at least 50 mm, and corners at which the longitudinal edges and transverse edges meet,
the metal sheet has a first electrical contact face and a second electrical contact face, which are connected to a current or voltage source, wherein
the first electrical contact face lies completely within a face consisting of the intersecting faces of the front side, the rear side, and the side faces with an imaginary first cylinder, wherein the cylinder axis of the imaginary first cylinder is perpendicular to the front side of the metal sheet and passes through one of the corners of the front side, the cylinder jacket of the imaginary first cylinder intersects the longitudinal edge of the front side at a distance a1 from the cylinder axis and intersects the transverse edge of the front side at a distance b1 from the cylinder axis, wherein
$$a 1 \leq 0.2 \times {L}_{longitudinaledge} and b 1 \leq 0.2 \times {L}_{transverseedge}$$
wherein
L_{longitudinaledge} is the length of the longitudinal edge and L_{transverseedge} is the length of the transverse edge, which meet at the corner through which the cylinder axis of the imaginary first cylinder passes,
the second electrical contact face lies completely within a face consisting of the intersecting faces of the front side, the rear side, and the side faces with an imaginary second cylinder, wherein the cylinder axis of the imaginary second cylinder is perpendicular to the front side and passes through the corner of the front side that is at the greatest distance from the corner through which the cylinder axis of the imaginary first cylinder passes, the cylinder jacket of the imaginary second cylinder intersects the longitudinal edge of the front side at a distance a2 from the cylinder axis and intersects the transverse edge of the front side at a distance b2 from the cylinder axis, wherein
$$a 2 \leq 0.2 \times {L}_{longitudinaledge} and b 2 \leq 0.2 \times {L}_{transverseedge}$$
wherein
L_{longitudinaledge} is the length of the longitudinal edge and L_{transverseedge} is the length of the transverse edge, which meet at the corner through which the cylinder axis of the imaginary second cylinder passes,
the metal sheet has a third electrical contact face and a fourth electrical contact face, which are connected to a voltage measurement device when the first and second electrical contact faces are connected to a current source, or which are connected to a current measurement device when the first and second electrical contact faces are connected to a voltage source,
the metal sheet, which is connected to the current or voltage source via its first and second electrical contact faces and to the voltage or current measurement device via its third and fourth electrical contact faces, is thermally treated so that oxide particles are formed in the metal sheet, and
during the thermal treatment, the electrical resistance or an electrical variable of the metal sheet proportional to the electrical resistance is determined.

2. The method according to claim 1, wherein the first and second electrical contact faces are on opposite side faces of the metal sheet.

3. The method according to claim 1, wherein the third and fourth electrical contact faces are at a distance from one another that is smaller than the distance between the first and second electrical contact faces; and/or wherein the third and fourth electrical contact faces are located outside of the intersecting faces of the front side, the rear side, and the side faces of the metal sheet with the imaginary first and second cylinders.

4. The method according to claim 1, wherein the third and fourth electrical contact faces and the first electrical contact face are on the same side face of the metal sheet, and the second electrical contact face is on another side face, preferably the opposite side face, of the metal sheet.

5. The method according to claim 1, wherein the first or second electrical contact face is contacted with an electrical line of the current or voltage source, and the third and fourth electrical contact faces are contacted with the electrical lines of the voltage or current measurement device under the effect of gravity of the metal sheet by placing the metal sheet on the electrical lines via its front side, rear side, or one of its side faces.

6. A method for measuring an electrical variable of a metal-containing shaped body during thermal dispersion hardening of the metal-containing shaped body, wherein
the metal-containing shaped body is a metal tube or a metal rod, wherein the metal tube or the metal rod
has a length L_{shapedbody} of at least 50 mm and a diameter D_{shapedbody} in the range from 10 mm to 300 mm,
ends at one of its ends with a first end face and at its opposite end with a second end face, the first and second end faces are connected to one another by a lateral face, the first end face and the second end face are each delimited by an outer boundary line,
the metal tube or the metal rod has a first electrical contact face and a second electrical contact face, and these electrical contact faces are connected to a current or voltage source, wherein
the first electrical contact face lies completely within a face consisting of the intersecting faces of the lateral face and the first end face with an imaginary first ellipsoid, wherein the imaginary first ellipsoid has a center point and semi-axes a1, b1 and c1, wherein the center point lies on the outer boundary line of the first end face of the metal tube or metal rod, the semi-axis a1 is parallel to the longitudinal axis of the metal tube or metal rod and has a length Lₐ₁≤0.2×L_{shapedbody}, and the semi-axes b1 and c1 have a length L_{b1}=L_{c1}≤0.4×D_{shapedbody},
the second electrical contact face lies completely within a face consisting of the intersecting faces of the lateral face and the second end face with an imaginary second ellipsoid, wherein the imaginary second ellipsoid has a center point and semi-axes a2, b2 and c2, the center point of the imaginary second ellipsoid is the point farthest from the center point of the imaginary first ellipsoid on the outer boundary line of the second end face of the metal tube or metal rod, the semi-axis a2 is parallel to the longitudinal axis of the metal tube or metal rod and has a length Lₐ₂≤0.2×L_{shapedbody}, and the semi-axes b2 and c2 have a length L_{b2}=L_{c2}≤0.4×D_{shapedbody},
the metal tube or the metal rod has a third electrical contact face and a fourth electrical contact face, and these electrical contact faces are connected to a voltage measurement device when the first and second electrical contact faces are connected to a current source, or are connected to a current measurement device when the first and second electrical contact faces are connected to a voltage source,
the metal tube or the metal rod, which is connected to the current or voltage source via its first and second electrical contact faces and to the voltage or current measurement device via its third and fourth electrical contact faces, is thermally treated so that oxide particles are formed in the metal tube or metal rod, and
during the thermal treatment of the metal tube or metal rod, its electrical resistance or an electrical variable proportional to the electrical resistance is determined.

7. The method according to claim 6, wherein the diameter D_{shapedbody} of the metal tube is its outer diameter, and the first and second end faces of the metal tube in each case have an inner boundary line in addition to the outer boundary line.

8. The method according to claim 6, wherein the third and fourth electrical contact faces are at a distance from one another that is smaller than the distance between the first and second electrical contact faces; and/or wherein the third and fourth electrical contact faces are located outside of the intersecting faces of the end faces and the lateral face with the imaginary first and second ellipsoids.

9. The method according to claim 6, wherein the first or second electrical contact face is contacted with an electrical line of the current or voltage source, and the third and fourth electrical contact faces are contacted with the electrical lines of the voltage or current measurement device under the effect of gravity of the metal rod or metal tube by placing the metal rod or the metal tube with its lateral face or one of its end faces on the electrical lines.

10. The method according to claim 1, wherein the metal from which the metal sheet, the metal rod or the metal tube is made is a noble metal alloy containing one or more non-noble metals.

11. The method according to claim 10, wherein the non-noble metal is selected from zirconium, cerium, scandium and yttrium and is present in the noble metal alloy in a total concentration of not more than 1 wt. %, and the balance is one or more noble metals selected from ruthenium, rhodium, gold, palladium, platinum, iridium and osmium, and unavoidable impurities.

12. The method according to claim 1, wherein the thermal treatment of the metal sheet, metal tube or metal rod takes place at a temperature of at least 750° C.

13. The method according to claim 1, wherein the thermal treatment of the metal sheet, metal tube or metal rod is terminated when the electrical resistance or the electrical variable of the metal sheet, metal tube or metal rod proportional thereto has a constant value over a defined minimum duration.

14. A measurement arrangement, containing
a current or voltage source,
a voltage or current measurement device,
a metal-containing shaped body, wherein the metal-containing shaped body is a metal sheet having a front side, a rear side opposite the front side, and side faces that connect the front side and the rear side to one another, wherein
the front side and the rear side in each case have longitudinal edges of which the length L_{longitudinaledge} is at least 50 mm, transverse edges of which the length L_{transverseedge} is at least 50 mm, and corners at which the longitudinal edges and transverse edges meet,
the metal sheet has a first electrical contact face and a second electrical contact face, which are connected to a current or voltage source, wherein
the first electrical contact face lies completely within a face consisting of the intersecting faces of the front side, the rear side, and the side faces with an imaginary first cylinder, wherein the cylinder axis of the imaginary first cylinder is perpendicular to the front side of the metal sheet and passes through one of the corners of the front side, the cylinder jacket of the imaginary first cylinder intersects the longitudinal edge of the front side at a distance a1 from the cylinder axis and intersects the transverse edge of the front side at a distance b1 from the cylinder axis, wherein
$$a 1 \leq 0.2 \times {L}_{longitudinaledge} and b 1 \leq 0.2 \times {L}_{transverseedge}$$
wherein
L_{longitudinaledge} is the length of the longitudinal edge and L_{transverseedge} is the length of the transverse edge, which meet at the corner through which the cylinder axis of the imaginary first cylinder passes,
the second electrical contact face lies completely within a face consisting of the intersecting faces of the front side, the rear side, and the side faces with an imaginary second cylinder, wherein the cylinder axis of the imaginary second cylinder is perpendicular to the front side and passes through the corner of the front side that is at the greatest distance from the corner through which the cylinder axis of the imaginary first cylinder passes, the cylinder jacket of the imaginary second cylinder intersects the longitudinal edge of the front side at a distance a2 from the cylinder axis and intersects the transverse edge of the front side at a distance b2 from the cylinder axis, wherein
$$a 2 \leq 0.2 \times {L}_{longitudinaledge} and b 2 \leq 0.2 \times {L}_{transverseedge}$$
wherein
L_{longitudinaledge} is the length of the longitudinal edge and L_{transverseedge} is the length of the transverse edge, which meet at the corner through which the cylinder axis of the imaginary second cylinder passes,
the metal sheet has a third electrical contact face and a fourth electrical contact face, and these electrical contact faces are connected to the voltage measurement device when the first and second electrical contact faces are connected to the current source, or are connected to the current measurement device when the first and second electrical contact faces are connected to the voltage source.

15. A measurement arrangement, containing
a current or voltage source,
a voltage or current measurement device,
a metal-containing shaped body, wherein the metal-containing shaped body is a metal tube or a metal rod, wherein the metal tube or the metal rod
has a length L_{shapedbody} of at least 50 mm and a diameter D_{shapedbody} in the range from 10 mm to 300 mm,
ends at one of its ends with a first end face and at its opposite end with a second end face, the end faces are connected to one another by a lateral face and are each delimited by an outer boundary line,
the metal tube or the metal rod has a first electrical contact face and a second electrical contact face, which are connected to the current or voltage source, wherein
the first electrical contact face lies completely within a face consisting of the intersecting faces of the lateral face and the first end face with an imaginary first ellipsoid, wherein the imaginary first ellipsoid has a center point and semi-axes a1, b1 and c1, wherein the center point lies on the outer boundary line of the first end face of the metal tube or metal rod, the semi-axis a1 is parallel to the longitudinal axis of the metal tube or metal rod and has a length Lₐ₁≤0.2×L_{shapedbody}, and the semi-axes b1 and c1 have a length L_{b1}=L_{c1}≤0.4×D_{shapedbody},
the second electrical contact face lies completely within a face consisting of the intersecting faces of the lateral face and the second end face with an imaginary second ellipsoid, wherein the imaginary second ellipsoid has a center point and semi-axes a2, b2 and c2, the center point of the imaginary second ellipsoid is the point farthest from the center point of the imaginary first ellipsoid on the outer boundary line of the second end face of the metal tube or metal rod, the semi-axis a2 is parallel to the longitudinal axis of the metal tube or metal rod and has a length Lₐ₂≤0.2×L_{shapedbody}, and the semi-axes b2 and c2 have a length L_{b2}=L_{c2}≤0.4×D_{shapedbody},
the metal tube or the metal rod has a third electrical contact face and a fourth electrical contact face, and these electrical contact faces are connected to a voltage measurement device when the first and second electrical contact faces are connected to a current source, or are connected to a current measurement device when the first and second electrical contact faces are connected to a voltage source.

16. A use of the measurement arrangement according to claim 14 for determining the duration of thermal dispersion hardening of the metal sheet, metal rod or metal tube.
